(19) United States
(12) Patent Application Publication
(10) Pub. No.: US 2026/0325813 A1
(43) Pub. Date: Oct. 22, 2026

(54) SYSTEM FOR CONNECTING A TUBE SET TO A SURGICAL ACCESS DEVICE

(71) Applicant: Conmed Corporation, Largo, FL (US)

(72) Inventors: Derek Eilers, Denver, CO (US); Mahesh Krishnamoorthy, Parker, CO (US)

(73) Assignee: Conmed Corporation, Largo, FL (US)

(21) Appl. No.: 19/110,623

(22) PCT Filed: Sep. 15, 2023
(86) PCT No.: PCT/US2023/032922

§ 371 Date: Mar. 11, 2025

Related U.S. Application Data
(60) Provisional application No. 63/407,365, filed on Sep. 16, 2022.

(51) Int. Cl. A61M 39/10; A61M 39/02

(52) U.S. Cl. CPC A61M 39/105; A61M 39/02; A61M 2039/1044; A61M 2205/43; A61M 2205/44; A61M 2205/581; A61M 2205/583

(57) ABSTRACT

In accordance with at least one aspect of this disclosure, a coupling system includes a multi-lumen surgical access device having a housing that includes a fitting, the fitting having a plurality of coaxial flow passages defined therein by a plurality of concentric annular walls, the coupling system including a coupling operatively associated with a multi-lumen tub set, the coupling including a generally cylindrical body having a first end portion that includes a plurality of concentric annular walls that are dimensioned for intimate engagement with the coaxial flow passages of the fitting, and a second end portion adapted and configured for magnetic attachment to the fitting to secure the multi-lumen tube set to the multi-lumen surgical access device.

### CROSS-REFERENCE TO RELATED APPLICATIONS

This application claims the benefit of priority to U.S. Provisional Patent Application No. 63/407,365, filed Sep. 16, 2022, the entire content of which is herein incorporated by reference.

### TECHNICAL FIELD

The present disclosure relates to surgical devices, and more particularly, to connectors for connecting a filtered tube set to a surgical access device for use during endoscopic or laparoscopic surgical procedures.

### BACKGROUND OF THE DISCLOSURE

Laparoscopic or “minimally invasive” surgical techniques are becoming commonplace in the performance of procedures such as cholecystectomies, appendectomies, hernia repair and nephrectomies. Benefits of such procedures include reduced trauma to the patient, reduced opportunity for infection, and decreased recovery time. Such procedures within the abdominal (peritoneal) cavity are typically performed through a surgical access device (such as a trocar or cannula, for example), which facilitates the introduction of laparoscopic instruments into the abdominal cavity of a patient.

Additionally, such procedures commonly involve filling or “insufflating” the abdominal (peritoneal) cavity with a pressurized fluid, such as carbon dioxide, to create what is referred to as a pneumoperitoneum. The insufflation can be carried out by a surgical access device equipped to deliver insufflation fluid, or by a separate insufflation device, such as an insufflation (Veress) needle. Introduction of surgical instruments into the pneumoperitoneum without a substantial loss of insufflation gas is desirable, in order to maintain the pneumoperitoneum.

During typical laparoscopic procedures, a surgeon makes three to four small incisions, usually no larger than about twelve millimeters each, which are typically made with the surgical access devices themselves, typically using a separate inserter or obturator placed therein. Following insertion, the inserter is removed, and the access device allows access for instruments to be inserted into the abdominal cavity.

The surgical access device must provide a means to maintain the pressure within the cavity by sealing between the trocar and the surgical instrument being used, while still allowing at least a minimum freedom of movement of the surgical instruments. Such instruments can include, for example, scissors, grasping instruments, and occluding instruments, cauterizing units, cameras, light sources and other surgical instruments. Sealing elements or mechanisms are typically provided on trocars to prevent the escape of insufflation gas, however, SurgiQuest, Inc., a wholly owned subsidiary of ConMed Corporation has developed unique gas sealed surgical access devices that permit ready access to an insufflated surgical cavity without the need for conventional mechanical valve seals, as described, for example, in U.S. Pat. No. 7,854,724 which is incorporated herein by reference in its entirety. These devices are constructed from several nested components including an inner tubular body portion and a coaxial outer tubular body portion. A tube set is used to provide surgical gas from a surgical gas delivery system to the surgical access device, e.g., a gas sealed surgical access device as in U.S. Pat. Nos. 7,854,724, 8,795,223 to SurgiQuest, or U.S. Pat. No. 11,026,717 to ConMed, all of which are incorporated by reference herein in its entirety.

The mechanical connection between the tube set, whether single lumen or multi-lumen, and the surgical access device or trocar is typically a threaded connection and often requires precise alignment, for example of a plurality of flow paths. Certain embodiments of mechanical connections between the tube set and surgical access device are described in U.S. Pat. Nos. 9,526,886, 10,960,197, and 11,065,430, all assigned to SurgiQuest, Inc. and all of which are incorporated by reference herein, In some instances, achieving a proper connection can be difficult and can require some level of dexterity. It would be beneficial therefore to provide a coupling system for detachably connecting a tube set, in particular a multi-lumen tube set, to a surgical access device that overcomes these difficulties.

### SUMMARY OF THE DISCLOSURE

In accordance with at least one aspect of this disclosure, a system, e.g., for connecting a tube set to a surgical access device, includes, a coupling operatively associated with a distal end portion of a tube set, the coupling having a first annular wall defining a first annulus about a first axis extending through the coupling. A fitting is operatively associated with a fluid port of a surgical access device, the fitting having a second annular wall defining a second annulus about a second axis extending through the coupling. The second annulus is configured for fluid communication with the first annulus when the coupling coupled to the fitting to provide a fluid from the tube set to the surgical access device through the fluid port of the surgical access device.

The coupling includes a first magnetic body radially outward from the first annular wall and the fitting includes a second magnetic body radially outward from the second annular wall. The first magnetic body and the second magnetic body are configured to interface with one another to couple the distal end portion of the tube set to the fluid port of the surgical access device and to axially secure the coupling to the fitting, while allowing for relative rotation of the coupling and the fitting about the first and second axes.

In embodiments, the coupling includes a first flange extending radially outward from the first annular wall and the first magnetic body is disposed in the first flange, e.g., the first flange defines an interior space configured to house at least a portion of the first magnetic body. In certain embodiments, the first magnetic body can be disposed entirely within the interior space of the first flange. In certain embodiments. The first magnetic body can be disposed at least partially within the interior space of the first flange such that a distal face of the first magnetic body is flush with a distal face of the first flange.

In embodiments, the fitting includes a second flange extending radially outward from the second annular wall and the second magnetic body is disposed in the second flange, e.g., the second flange defines an interior space configured to house at least a portion of the second magnetic body. In certain embodiments, the second magnetic body can be disposed entirely within the interior space of the first flange. In certain embodiments, the second magnetic body can be disposed at least partially within the interior space of the second flange such that a distal face of the second magnetic body is flush with a distal face of the second flange.

In embodiments, a signal indication device is disposed on the fitting and operatively connected to determine if the first and second magnetic bodies are properly interfaced. The signal indication device is configured to provide an audible or visual indication that the coupling and fitting are properly interfaced based on a position of the first and second magnetic bodies relative to one another. The signal indication device is configured to issue a first signal notification to a user to indicate the first and second magnetic bodies are properly interfaced and a second signal notification to the user to indicate the first and second magnetic bodies are not properly interfaced, and, in embodiments, the first signal notification is different from the second signal notification.

In certain embodiments, the indicator device can be or include an LED lamp configured to illuminate a first color as the first signal notification and illuminate a second color different than the first color as the second signal notification. In embodiments, the LED lamp can illuminate the same color, but in different strobe patterns for the first and second signal notifications. In certain embodiments, the indicator device can be or include a speaker configured to issue a first sound as the first signal notification and a second sound as the second signal notification, the second sound being different from the first sound.

In certain embodiments, the first magnetic body can be an arcuate magnetic body curving about the first axis, and the second magnetic body can be an arcuate magnetic body curving about the second axis. In certain such embodiments, the signal indication device is configured to issue the first signal notification to the user when the first magnetic body is interfaced with and circumferentially aligned with the second magnetic body. The signal indication device is configured to issue the second signal notification to the user when the first magnetic body is not interfaced with second magnetic body and/or when the first magnetic body is not circumferentially aligned with the second magnetic body. In such embodiments, circumferentially aligning the first magnetic body with the second magnetic body may include partially interfacing the first magnetic body with the second magnetic body and rotating the coupling or fitting relative to the other to algin the first magnetic body with the second magnetic body until the indicator device issues the first signal notification. In embodiments, the first magnetic body and second magnetic body may be disposed in the respective flange such that rotation is required to properly interface the first and second magnetic bodies.

In certain embodiments, the first magnetic body can include a first plurality of magnetic bodies arranged circumferentially about the first axis and wherein the second magnetic body can include a second plurality of magnetic bodies arranged circumferentially about the second axis. In certain such embodiments, the signal indication device can be configured to issue the first signal notification to the user when each magnetic body of the first plurality of magnetic bodies is interfaced with and circumferentially aligned with the each magnetic body of the second plurality of magnetic bodies. The signal indication device is configured to issue the second signal notification to the user when each magnetic body of the first plurality of magnetic bodies is not interfaced with each magnetic body of the second plurality of magnetic bodies and/or when each magnetic body of the first plurality of magnetic bodies is not circumferentially aligned with each magnetic body of the second plurality of magnetic bodies. Here, properly interfacing the magnets of the coupling and the magnets of the fitting may not require any rotation, so long as each respective magnet in the coupling is interfaced with a respective magnet in the fitting.

In accordance with at least one aspect of this disclosure, a system includes a tube set having at least one lumen, the at least one lumen including a tube set coupling at a distal end portion thereof having a first annular wall defining a first annulus about a first axis extending through the coupling, the tube set coupling including a first securing flange extending radially outward from the first annular wall having a first magnet therein. The system includes a surgical access device having a fitting operatively associated with a gas port of the surgical access device configured to be coupled to the tube set with the tube set coupling, the gas port having a second annular wall defining a second annulus about a second axis extending through the fitting, the gas port including a second securing flange extending radially outward from the second annular wall having a second magnet therein.

In embodiments, the first securing flange and the second securing flange are configured to interface with one another to couple the tube set coupling to the fitting of the fluid port to establish a first flow path from the at least one lumen through the first annulus to the second annulus and to the surgical access device through the fluid port of the surgical access device.

In certain embodiments, the tube set is a multi-lumen tube set having a plurality of lumens (e.g., at least two or three lumens), the plurality of lumens joining at the tube set coupling. The tube set coupling further includes a plurality of concentric annular walls radially inward of the first annular wall, defining a plurality of annuli, wherein each respective annulus is in fluid communication with a respective lumen. The first annular wall is a first outer annular wall, and the plurality of concentric annular walls includes the first outer annular wall, a first medial annular wall, and a first inner annular wall. The first annulus is a first outer annulus defined between the first outer annular wall and the first medial annular wall, a first medial annulus is defined between the first medial annular wall and the first inner annular wall, and a first inner annulus is defined within the first inner annular wall. The first outer annulus is in fluid communication with the first lumen defining the first flow path, the first medial annulus is in fluid communication with a second lumen defining a second flow path, and the first inner annulus is in fluid communication with a third lumen defining a third flow path.

The gas port of the surgical access device is a multi-lumen gas port, the fluid port further including a plurality of concentric annular walls radially inward of the second annular wall, defining a plurality of annuli, wherein each respective annulus is in fluid communication with a respective flow path, wherein the second annular wall is a second outer annular wall, wherein the plurality of concentric annular walls includes the second outer annular wall, a second medial annular wall, and a second inner annular wall, wherein the second annulus is a second outer annulus defined between the second outer annular wall and the second medial annular wall, a second medial annulus is defined between the second medial annular wall and the second inner annular wall, and a second inner annulus is defined within the second inner annular wall.

When the first securing flange is interfaced with the second securing flange, the first outer annulus is in fluid communication with the second outer annulus, the first medial annulus is in fluid communication with the second medial annulus, and the first inner annulus is in fluid communication with the second inner annulus. The first flow path, the second flow path, and the third flow path are configured to supply a gas from the tube set to the surgical access device or receive a gas from the surgical access device. Within the respective annuli and lumens, the first flow path, the second flow path, and the third flow path are fluidly isolated from one another.

In embodiments, a signal indication device can be disposed on the fitting and operatively connected to determine if the first and second magnets are properly interfaced and configured to provide an audible or visual indication that the coupling and fitting are properly interfaced based on a position of the first and second magnets relative to one another. The signal indication device is configured to issue a first signal notification to a user to indicate the first and second magnets are properly interfaced and a second signal notification to the user to indicate the first and second magnets are not properly interfaced. In embodiments, the first signal notification is different from the second signal notification.

The signal indication device is configured to issue the first signal notification to the user when the first magnet is interfaced with and circumferentially aligned with the second magnet and configured to issue the second signal notification to the user when the first magnet is not interfaced with second magnet and/or when the first magnet is not circumferentially aligned with the second magnet.

In accordance with at least one aspect of this disclosure, a coupling system includes a multi-lumen surgical access device having a housing that includes a fitting, the fitting having a plurality of coaxial flow passages defined therein by a plurality of concentric annular walls. The coupling system includes a coupling operatively associated with a multi-lumen tub set, the coupling including a generally cylindrical body having a first end portion that includes a plurality of concentric annular walls that are dimensioned for intimate engagement with the coaxial flow passages of the fitting, and a second end portion adapted and configured for magnetic attachment to the fitting to secure the multi-lumen tube set to the multi-lumen surgical access device.

### BRIEF DESCRIPTION OF THE DRAWINGS

So that those skilled in the art to which the subject disclosure appertains will readily understand how to make and use the devices and methods of the subject disclosure without undue experimentation, embodiments thereof will be described in detail herein below with reference to certain figures, wherein:

FIG. 1 is an illustration of a surgical arena during a laparoscopic procedure performed on a patient utilizing a multi-modal gas delivery device and an associated filtered tube set which includes a coupling system constructed in accordance with an exemplary embodiment of the subject invention;

FIG. 2 is a perspective view of an embodiment of a filtered tube set configured for interfacing with the gas delivery device of FIG. 1, which includes a filter cartridge having a tube set attached thereto and an embodiment of a coupling associated therewith shown with a surgical access device having an embodiment of a fitting associated therewith, the fitting and coupling configured for intimate engagement with one another;

FIG. 3 is an enlarged localized perspective view an embodiment of the fitting shown in FIG. 2;

FIG. 4 is an enlarged localized perspective view of an embodiment of the coupling shown in FIG. 2;

FIG. 5 is an enlarged perspective view of the an embodiment of the coupling and an embodiment of the fitting shown detached from one another;

FIGS. 6 through 8 illustrate the steps for engaging the coupling with the fitting of the surgical access device; and

FIG. 9 is an enlarged perspective view of the an embodiment of the coupling and an embodiment of the fitting shown detached from one another.

### DETAILED DESCRIPTION

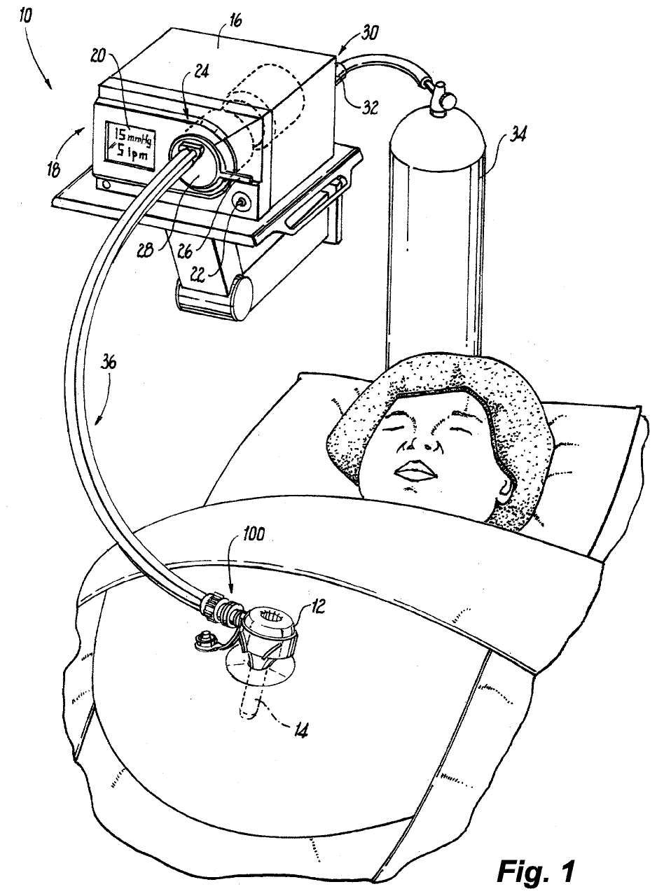

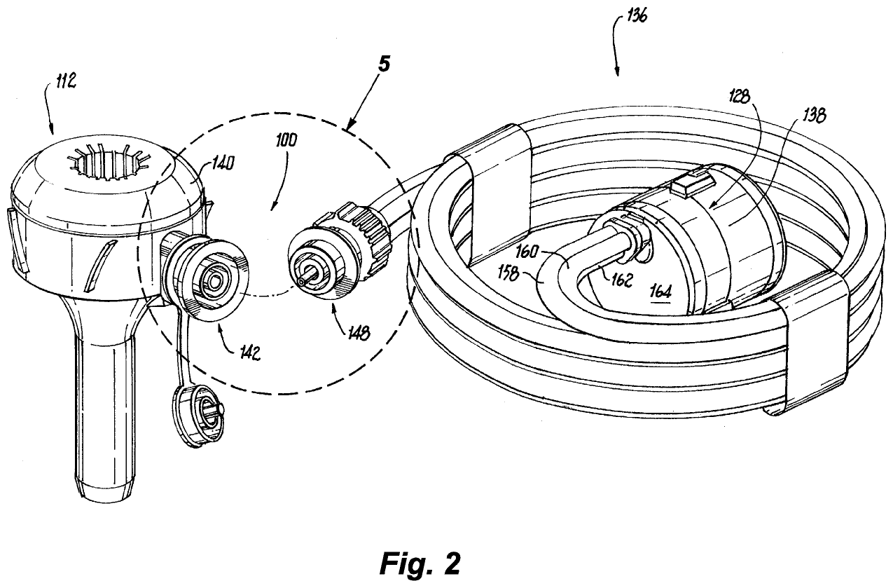

Reference will now be made to the drawings wherein like reference numerals identify similar structural features or aspects of the subject disclosure. For purposes of explanation and illustration, and not limitation, an illustrative view of an embodiment of a coupling system configured in accordance with the subject disclosure is shown in FIG. 1 and is designated generally by reference numeral 100. Other embodiments and/or aspects of this disclosure are shown in FIGS. 2-9.

There is illustrated in FIG. 1 a surgical gas delivery system 10 for use during endoscopic surgical procedures, where, in FIG. 1, the surgical gas delivery system 10 is shown coupled to a surgical access device 12 for delivering surgical gas to a surgical cavity 14. The system 10 includes a device housing 16. The front face 18 of the housing 16 has a capacitive or resistive touch screen 20 for presenting a graphical user interface (GUI) and a power switch 22 for turning the device 10 on and off.

The front face 18 of the housing 16 further includes a filter cartridge interface 24 with a rotatable latch mechanism 26 configured to facilitate the secure engagement of a disposable filter cartridge 28 within the device housing 16. The rear face 30 of the housing 16 includes a gas supply fitting 32 for connection with a source of compressed gas 34, a standard USB interface for service purposes, and a standard power connection (USB and power connection not shown). The filter cartridge 28 includes a tube set 36 extending there from to supply the surgical gas to the surgical cavity 14, through eh surgical access device 12. The filter cartridge 28 and the tube set 36 can be similar to that disclosed in U.S. Pat. Nos. 9,375,539 and 9,067,030, the entire contents of which are incorporated herein by reference.

In embodiments, the filtered tube set 36 described herein can be suitable for use with the surgical gas delivery device 10, e.g., as described above and as shown in FIG. 1. As shown in FIGS. 1 and 2, the filtered tube set 36 includes the filter cartridge 28 having a housing 38 defining an inlet flow path for receiving a flow of gas from a patient's abdominal cavity 14 and an outlet flow path for delivering a flow of gas to the patient's abdominal cavity 14. In embodiments, the inlet flow path of the filter cartridge and/or the outlet path of the filter cartridge 28 can include one or more filter elements therein.

With reference now to FIGS. 2-4, in accordance with at least one aspect of this disclosure, the coupling system 100 includes a multi-lumen surgical access device 112 having a housing 140 that includes a fitting 142, the fitting 142 having a plurality of coaxial flow passages 144a, 144b, 144c defined therein by a plurality of concentric annular walls 146a, 146b, 146c. The coupling system 100 includes a coupling 148 operatively associated with a multi-lumen tub set 136, the coupling 148 including a generally cylindrical body 150 having a first end portion 152 that includes a plurality of concentric annular walls 154a, 154b, 154c that are dimensioned for intimate engagement with the coaxial flow passages 144a, 144b, 144c of the fitting 142. The coupling 148 includes a second end portion 156 adapted and configured for magnetic attachment to the fitting 142 to secure the multi-lumen tube set 136 to the multi-lumen surgical access device 112.

The multi-lumen tube set 136 includes a first lumen 158, a second lumen 160, and a third lumen 162 extending from a front end 164 of the housing 138 of the filter cartridge 128 and meet at the coupling 148, where each of the first lumen 158, the second lumen 160, and the third lumen 162 meet a respective annulus of a plurality of annuli defined within the concentric annular walls 154a, 154b, 154c. For example, the coupling 148 includes flow passages 166a, 166b, 166c, which, when the coupling 148 is coupled to the fitting 142, are in fluid communication with the co-axial flow passages 144a, 144b, 144c. Each of the first lumen 158, the second lumen 160, and the third lumen 162 are configured for carrying a fluid (e.g., a gas such as surgical gas, or a gas/liquid mixture such as humidified surgical gas) to from the gas deliver system 10 to the surgical cavity 14 or from the surgical access device 112 to the gas delivery system 10.

For example, one lumen is configured for delivering surgical gas, e.g., insufflation gas, to the surgical cavity 14. This lumen can also be used for pressure sensing when insufflation gas is not flowing into the surgical cavity 14. One lumen is configured to provide a high pressure gas flow for forming an air seal within a jet assembly in the housing of the surgical access device 112. One lumen is configured for providing a recirculation flow path or a return flow path from the surgical access device 112, taking a low pressure flow from the surgical access device 112 and returning it to the gas delivery system 10. This lumen can also provide smoke evacuation from the surgical cavity 14 as the spent gas from the high pressure flow is returned to the gas delivery system 10. These features of the multi-lumen access device 112 are explained in more detail in U.S. Pat. No. 7,854,724, and are not discussed in detail herein.

With continued reference to FIG. 3, the fitting 142 includes a first annular wall 146a defining a first annulus 144a about a first axis A1 extending through the fitting 142. The first annular wall 146a can be an outer annular wall, and the first annulus can be an outer annulus. The fitting 142 includes a medial annular wall 146b, radially inward of the first annular wall 146a, bounding the first annulus 144a between the first annular wall 146a and the second annular wall 146b. The fitting 142 includes an inner annular wall 146c, which is radially inward of the medial annular wall 146b. The inner annular wall 146c and the medial annular wall bound the medial annulus 144b therebetween. The inner annular wall 146c forms an inner annulus 144c therein. The fitting 142 also includes a magnetic body 168 radially outward from the first annular wall 146a. The fitting 142 can include a securing flange 170 which extends radially outward from the first annular wall 146a and the magnetic body 166 can be disposed within the flange 170, on the flange 170, or partially within the flange 170 as discussed further below.

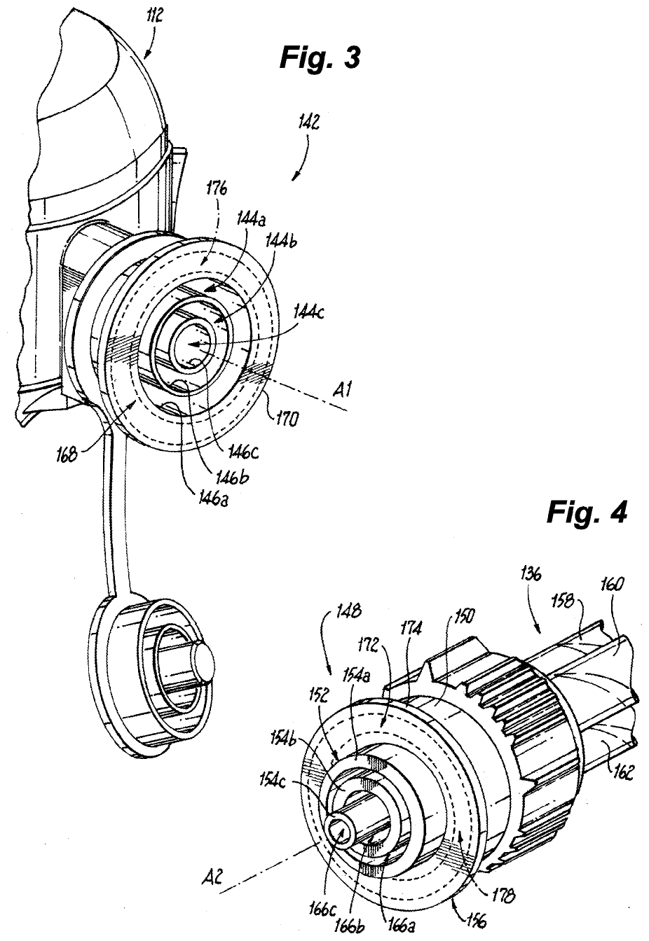

The coupling 148 includes similar features to that of the fitting 142, for example, as shown in FIG. 4, the coupling includes a first (outer) annular wall 154a and a medial annular wall 154b radially inward from the first annular wall 154a, where a first (outer) annulus 166a is defined therebetween. The coupling 148 includes an inner annular wall 154c radially inward of the medial annular wall 154b, and a medial annulus 166b is bound by the medial annular wall 154b and the inner annular wall 154c. The inner annular wall 154c an inner annulus 166c therein. When the coupling 148 and fitting 142 are connected, the flow passages 144a, 164a are in fluid communication to form a contiguous flow path between the gas delivery system 10 and the surgical access device 112, through the first lumen 158. The flow passages 144b, 164b are in fluid communication to form a contiguous flow path between the gas delivery system 10 and the surgical access device 112, through the second lumen 160. The flow passages 144c, 164c are in fluid communication to form a contiguous flow path between the gas delivery system 10 and the surgical access device 112, though the third lumen 162.

The coupling 148 includes a magnetic body 172 radially outward from the outer annular wall 154a. The coupling 148 can include a securing flange extending radially outward from the outer annular wall 154a, and the magnetic body 172 can be disposed within the flange 174, on the flange 174, or partially within the flange 174 as discussed further below. The magnetic bodies 168, 172 are configured to interface with one another to couple the distal end portion (e.g., the combined end portion 152, 156) of the tube set 136 to the fitting 142 of the surgical access device 112 and to axially secure the coupling 148 to the fitting 142. The magnetic attachment is configured to hold the coupling 148 to the fitting 142 axially, but still allow for relative rotation of the coupling 148 and the fitting 142 about the first and second axes A1/A2 when coupled.

Still with reference to FIGS. 3 and 4, the respective securing flanges and magnetic bodies will be discussed. As shown in FIGS. 3 and 4, the securing flanges 170, 174 can define respective interiors spaces 176, 178, and the magnetic body 168 can be disposed entirely within the securing flange 170 of the fitting 142 and the magnetic body 172 can be disposed entirely within the securing flange 174 of the coupling 148. Also, as shown in FIGS. 3 and 4, the magnetic bodies 168, 172 can be circumferentially shaped, and extend along an entirety of the circumference of the respective securing flange 170, 174.

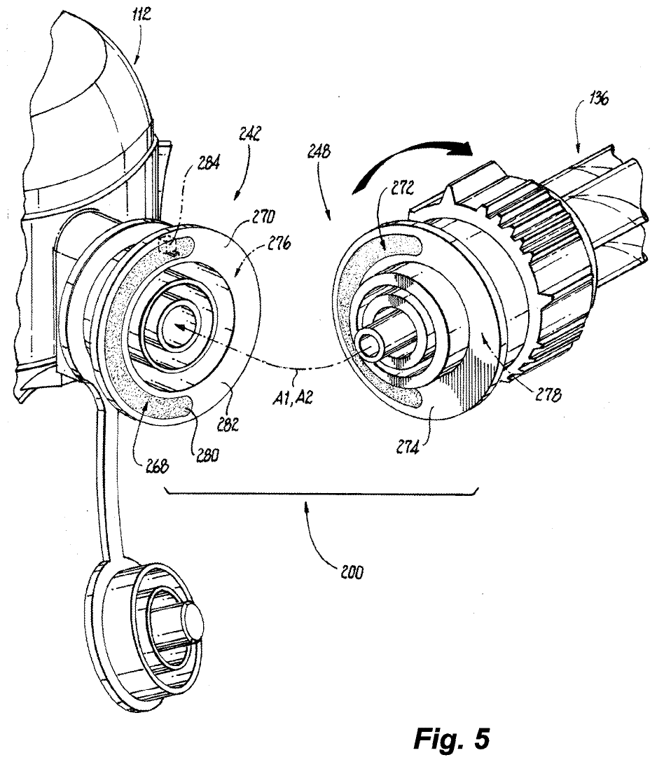

Turning now to FIGS. 5-8, in certain embodiments, as shown with respect to coupling system 200, the magnetic body 268 of the fitting 242 can can be disposed at least partially within the interior space 276 of the second flange 278 such that a distal face 280 of the magnetic body 268 is flush with a distal face 282 of the securing flange 270. The same can be true for the magnetic body 272 of the coupling 248, where the magnetic body 272 is shown partially within the interior space 278 of the securing flange 274.

A signal indication device 284 can be disposed on the fitting 242 and operatively connected (e.g., to at least the magnetic body 268) to determine if the magnetic body 268 is properly interfaced with the magnetic body 272. For example, the signal indication device 284 can be configured to provide an visual indication 286a and/or an audible indication 286b (e.g., as shown in FIG. 8) that the fitting 242 and coupling 248 are properly interfaced (e.g., sufficiently secured for the given surgical procedure) based on a position of the magnetic bodies 268, 272 relative to one another. The signal indication device 284 can be configured to issue a first signal notification (e.g., 286a) to a user to indicate the magnetic bodies 268, 272 are properly interfaced and a second signal notification (e.g., 286b) to the user to indicate the magnetic bodies 268, 272 are not properly interfaced. In certain embodiments, as shown, the first notification can be visual and the second notification can be audible for easy differentiation by the user.

In certain embodiments, the indicator device 284 can be or include an LED lamp configured to illuminate a first color as the first signal notification and illuminate a second color different than the first color as the second signal notification. In certain embodiments, the LED lamp can illuminate the same color, but in different strobe patterns for the first and second signal notifications. In certain embodiments, the indicator device 284 can be or include a speaker configured to issue a first sound as the first signal notification and a second sound as the second signal notification, the second sound being different from the first sound. Any suitable combination of audio/visual signal identification, e.g., including different colors, strobe patterns, tones, pitches, tone patters, etc. is contemplated herein.

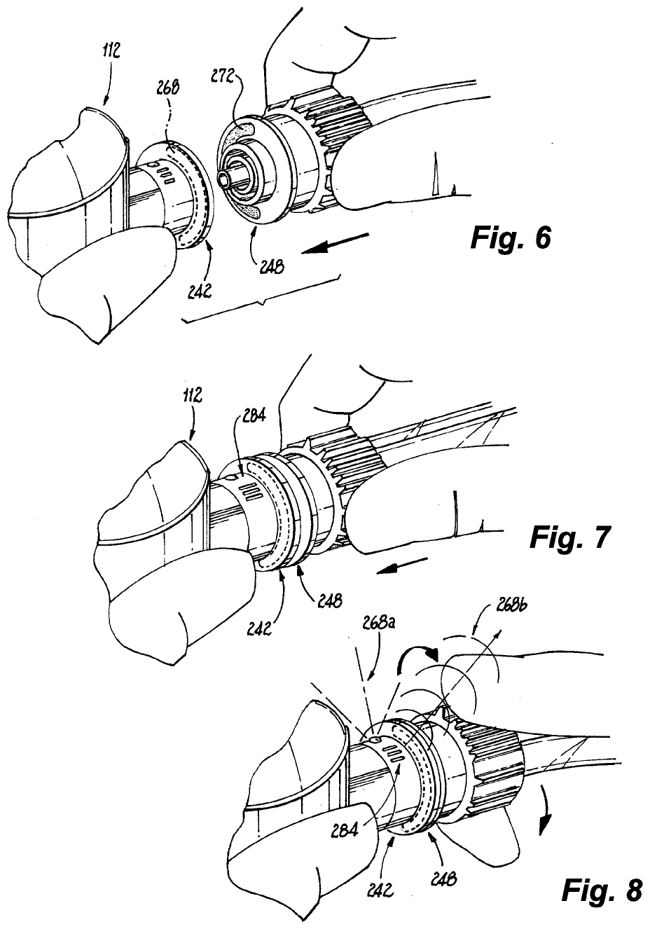

With continued reference to FIGS. 5-8, in certain embodiments, the magnetic bodies 268, 270 are arcuate, curving around the axis A1, A2. As shown, the magnetic bodies 268, 272 can be disposed in the respective flange 270, 274 on opposed sides so that relative rotation of one of the fitting 242 or coupling 248 is required for proper securement of the coupling 248 to the fitting 242. This is shown in the animation of FIGS. 6-8. In such embodiments, the signal indication device 284 can be configured to issue the first signal notification 286a to the user when the magnetic body 268 is interfaced with and circumferentially aligned with the magnetic body 272, e.g., such that the magnets 268, 272 are completely interfaced with one another. The signal indication device 284 can thus be configured to issue the second signal notification 286b to the user when the magnetic body 268 is either not interfaced with the magnetic body 272 at all, or when the magnetic body 268 is interfaced with the magnetic body 272, but they are out of alignment (e.g., where the relative rotation of the fitting 242 or coupling 248 has not yet occurred or is not yet complete). The signal indication device 284 can issue the second signal notification 286b to the user until the proper interfacing alignment is achieved, wherein the signal indication device 284 will issue the first signal notification 286a to the user. In this way, the signal indication device 284 can also operate to serve as a check, in the event the one of the fitting 242 or coupling 248 comes loose during the procedure, the user will be notified to check the connection between the tube set 136 and the surgical access device 112.

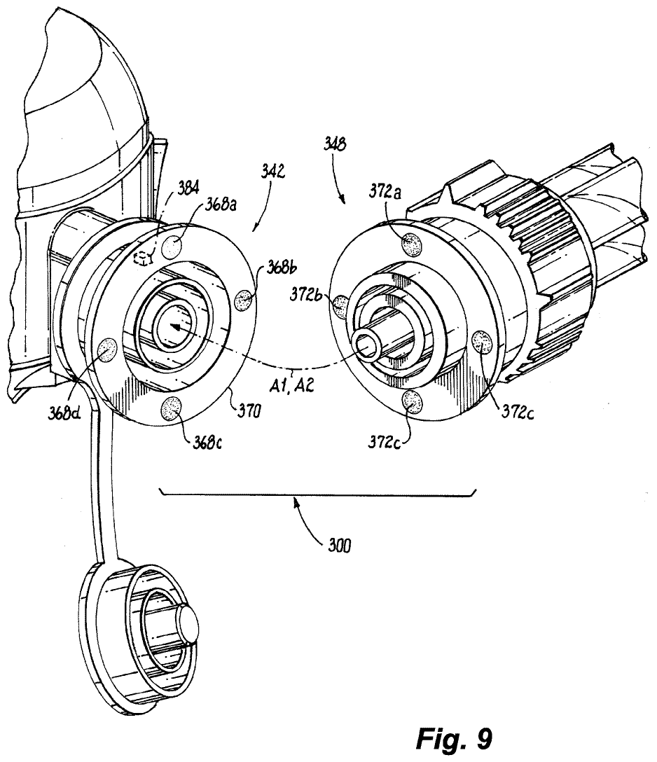

Referring now to FIG. 9, which shows another embodiment of a coupling system 300. The coupling system 300 can be similar to that of coupling system 200, and may include similar features, even if not expressly described herein below. As shown, the magnetic body of the fitting 342 can include a plurality of magnets 368a, 368b, 368c, and 368d disposed in the securing flange 370 and arranged circumferential to the axis A1. Similarly, the magnetic body of the fitting 348 can include a plurality of magnets 372a, 372b, 372c, and 372d disposed in the securing flange 374 and arranged circumferential to the axis A2.

The signal indication device 384 can be configured to issue the first signal notification 286a to the user when each magnet 368a, 368b, 368c, and 368d is interfaced with and circumferentially aligned with one of 372a, 372b, 372c, and 372d. It is not required that 368a align with 372a, for example, in order to be properly interfaced, but only that each magnet on the fitting 342 is fully interfaced with a respective magnet on the coupling 348. The signal indication device 384 can be configured to issue the second signal notification 286b to the user when each magnet 368a, 368b, 368c, and 368d is not interfaced with and/or not circumferentially aligned with one of 372a, 372b, 372c, and 372d. Here, properly interfacing the magnets of the coupling 348 and the magnets of the fitting 342 may not require any rotation.

In traditional connection or coupling systems, the attachment between an access device (e.g., an access port, a cannula, or trocar) and a tube set is a manual process of applying a force by a clinician. Such a manual process can lead to leaks at the junction which can, in instances go unnoticed. A leak at the junction can lead to loss of gas flow to the patient which may hinder achieving pneumoperitoneum or lead to of loss of pneumoperitoneum. Embodiments of the coupling system as provided herein allows for the access device and tube set connection itself to indicate whether the connection is proper, which can improve ease of connection and can minimize any leaks that may be caused at the junction.

To improve the connection between access port and tube set, magnets can be included into the manifold section of the access port and in the tube-set connector. The magnet strength can be selected to provide a proper seal, but not excessive, so that a clinician can disconnect with one hand. In embodiments, the magnets can create a linear mate or self-align with a turn. Embodiments as provided herein can enable the tube-set to be connected to the access port without the need for manually applying the torque and rotating. The torque and the force required will instead be determined by the magnets placed on the manifold of access port and the tube-set connector. Further, traditional systems may lack means to check the tightness and integrity of the connection. The magnets of the improved connectors will ensure that the tube set is always attached. The magnetic strength can also be adjusted so that the tube-set can be easily removed from the access port when needed.

In embodiments, the strength of the magnetic connection can also be linked to an indicator (e.g., an LED light) that can indicate the connection between the surgical device and the tube set is good or bad or that readjustment is needed. The indicator will provide confirmation at the beginning and during the surgical procedure that the connection between the tube set and manifold is “good” using a “green light,” or “bad” using a “red light,” or any other suitable light/sound color/pattern combination. Embodiments can also be applied to an existing luer lock connections for initial insufflation and for air seal robotic solution bifurcated lumen through means of couplers on both ends. Embodiments provide a coupling system that enables the tube set to be connected to the surgical access device without the need for manually applying the torque and rotating the coupling.

Those having ordinary skill in the art understand that any numerical values disclosed herein can be exact values or can be values within a range. Further, any terms of approximation (e.g., “about”, “approximately”, “around”) used in this disclosure can mean the stated value within a range. For example, in certain embodiments, the range can be within (plus or minus) 20%, or within 10%, or within 5%, or within 2%, or within any other suitable percentage or number as appreciated by those having ordinary skill in the art (e.g., for known tolerance limits or error ranges).

Any suitable combination(s) of any disclosed embodiments and/or any suitable portion(s) thereof are contemplated herein as appreciated by those having ordinary skill in the art in view of this disclosure.

The embodiments of the present disclosure, as described above and shown in the drawings, provide for improvement in the art to which they pertain. While the apparatus and methods of the subject disclosure have been shown and described, those skilled in the art will readily appreciate that changes and/or modifications may be made thereto without departing from the scope of the subject disclosure.

What is claimed is:

1. A system for connecting a tube set to a surgical access device, comprising:
a coupling operatively associated with a distal end portion of a tube set, the coupling having a first annular wall defining a first annulus about a first axis extending through the coupling; and
a fitting operatively associated with a fluid port of a surgical access device, the fitting having a second annular wall defining a second annulus about a second axis extending through the coupling, the second annulus configured for fluid communication with the first annulus when the is coupling coupled to the fitting;
wherein the coupling includes a first magnetic body radially outward from the first annular wall,
wherein the fitting includes a second magnetic body radially outward from the second annular wall, and
wherein the first magnetic body and the second magnetic body are configured to interface with one another to couple the distal end portion of the tube set to the fluid port of the surgical access device and to axially secure the coupling to the fitting, while allowing for relative rotation of the coupling and the fitting about the first and second axes.

2. The system of claim 1, wherein the coupling includes a first flange extending radially outward from the first annular wall, wherein the first magnetic body is disposed in the first flange.

3. The system of claim 2, wherein the first flange defines an interior space configured to house at least a portion of the first magnetic body, wherein the first magnetic body is disposed entirely within the interior space of the first flange.

4. The system of claim 2, wherein the first magnetic body is disposed at least partially within the interior space of the first flange such that a distal face of the first magnetic body is flush with a distal face of the first flange.

5. The system of claim 1, wherein the fitting includes a second flange extending radially outward from the second annular wall, wherein the second magnetic body is disposed in the second flange.

6. The system of claim 5, wherein the second flange defines an interior space configured to house at least a portion of the second magnetic body, wherein the second magnetic body is disposed entirely within the interior space of the first flange.

7. The system of claim 5, wherein the second magnetic body is disposed at least partially within the interior space of the second flange such that a distal face of the second magnetic body is flush with a distal face of the second flange.

8. The system of claim 1, further comprising signal indication device disposed on the fitting and operatively connected to determine if the first and second magnetic bodies are properly interfaced and configured to provide an audible or visual indication that the coupling and fitting are properly interfaced based on a position of the first and second magnetic bodies relative to one another.

9. The system of claim 8, wherein the signal indication device is configured to issue a first signal notification to a user to indicate the first and second magnetic bodies are properly interfaced and a second signal notification to the user to indicate the first and second magnetic bodies are not properly interfaced, wherein the first signal notification is different from the second signal notification.

10. The system of claim above, wherein the first magnetic body is an arcuate magnetic body curving about the first axis, and wherein the second magnetic body is an arcuate magnetic body curving about the second axis,
wherein the signal indication device is configured to issue the first signal notification to the user when the first magnetic body is interfaced with and circumferentially aligned with the second magnetic body, and wherein the signal indication device is configured to issue the second signal notification to the user when the first magnetic body is not interfaced with second magnetic body and/or when the first magnetic body is not circumferentially aligned with the second magnetic body.

11. The system of claim 9, wherein the first magnetic body includes a first plurality of magnetic bodies arranged circumferentially about the first axis and wherein the second magnetic body includes a second plurality of magnetic bodies arranged circumferentially about the second axis,
wherein the signal indication device is configured to issue the first signal notification to the user when each magnetic body of the first plurality of magnetic bodies is interfaced with and circumferentially aligned with the each magnetic body of the second plurality of magnetic bodies and wherein the signal indication device is configured to issue the second signal notification to the user when each magnetic body of the first plurality of magnetic bodies is not interfaced with each magnetic body of the second plurality of magnetic bodies and/or when the each magnetic body of the first plurality of magnetic bodies is not circumferentially aligned with each magnetic body of the second plurality of magnetic bodies.

12. The system of claim 1, wherein the first magnetic body is an arcuate magnetic body curving about the first axis and wherein the second magnetic body is an arcuate magnetic body curving about the second axis.

13. The system of claim 1, wherein the first magnetic body includes a first plurality of magnetic bodies arranged circumferentially about the first axis and wherein the second magnetic body includes a second plurality of magnetic bodies arranged circumferentially about the second axis.

14. A system, comprising:
a tube set having at least one lumen, the at least one lumen including a tube set coupling at a distal end portion thereof having a first annular wall defining a first annulus about a first axis extending through the coupling, the tube set coupling including a first securing flange extending radially outward from the first annular wall having a first magnet therein; and
a surgical access device having a fitting operatively associated with a gas port of the surgical access device configured to be coupled to the tube set with the tube set coupling, the gas port having a second annular wall defining a second annulus about a second axis extending through the fitting, the gas port including a second securing flange extending radially outward from the second annular wall having a second magnet therein,
wherein the first securing flange and the second securing flange are configured to interface to couple the tube set coupling to the fitting of the fluid port to establish a first flow path from the at least one lumen through the first annulus to the second annulus and to the surgical access device through the fluid port of the surgical access device.

15. The system of claim 14, wherein the tube set is a multi-lumen tube set having a plurality of lumens, the plurality of lumens joining at the tube set coupling, the tube set coupling further including a plurality of concentric annular walls radially inward of the first annular wall, defining a plurality of annuli, wherein each respective annulus is in fluid communication with a respective lumen, wherein the first annular wall is a first outer annular wall, wherein the plurality of concentric annular walls includes the first outer annular wall, a first medial annular wall, and a first inner annular wall, wherein the first annulus is a first outer annulus defined between the first outer annular wall and the first medial annular wall, a first medial annulus is defined between the first medial annular wall and the first inner annular wall, and a first inner annulus is defined within the first inner annular wall, wherein the first outer annulus is in fluid communication with the first lumen defining the first flow path, the first medial annulus is in fluid communication with a second lumen defining a second flow path, and the first inner annulus is in fluid communication with a third lumen defining a third flow path.

16. The system of claim 15, wherein the gas port of the surgical access device is a multi-lumen gas port, the fluid port further including a plurality of concentric annular walls radially inward of the second annular wall, defining a plurality of annuli, wherein each respective annulus is in fluid communication with a respective flow path, wherein the second annular wall is a second outer annular wall, wherein the plurality of concentric annular walls includes the second outer annular wall, a second medial annular wall, and a second inner annular wall, wherein the second annulus is a second outer annulus defined between the second outer annular wall and the second medial annular wall, a second medial annulus is defined between the second medial annular wall and the second inner annular wall, and a second inner annulus is defined within the second inner annular wall.

17. The system of claim 16, wherein, when the first securing flange is interfaced with the second securing flange, the first outer annulus is in fluid communication with the second outer annulus, the first medial annulus is in fluid communication with the second medial annulus, and the first inner annulus is in fluid communication with the second inner annulus, wherein the first flow path, the second flow path, and the third flow path are configured to supply a gas from the tube set to the surgical access device or receive a gas from the surgical access device, wherein the first flow path, the second flow path, and the third flow path are fluidly isolated from one another.

18. The system of claim 15, further comprising signal indication device disposed on the fitting and operatively connected to determine if the first and second magnets are properly interfaced and configured to provide an audible or visual indication that the coupling and fitting are properly interfaced based on a position of the first and second magnets relative to one another,
wherein the signal indication device is configured to issue a first signal notification to a user to indicate the first and second magnets are properly interfaced and a second signal notification to the user to indicate the first and second magnets are not properly interfaced, wherein the first signal notification is different from the second signal notification.

19. The system of claim 18, wherein the signal indication device is configured to issue the first signal notification to the user when the first magnet is interfaced with and circumferentially aligned with the second magnet and wherein the signal indication device is configured to issue the second signal notification to the user when the first magnet is not interfaced with second magnet and/or when the first magnet is not circumferentially aligned with the second magnet.

20. A coupling system comprising:
a) a multi-lumen surgical access device having a housing that includes a fitting, the fitting having a plurality of coaxial flow passages defined therein by a plurality of concentric annular walls; and
b) a coupling operatively associated with a multi-lumen tub set, the coupling including a generally cylindrical body having a first end portion that includes a plurality of concentric annular walls that are dimensioned for intimate engagement with the coaxial flow passages of the fitting, and a second end portion adapted and configured for magnetic attachment to the fitting to secure the multi-lumen tube set to the multi-lumen surgical access device.
